(19) United States
(12) Patent Application Publication
(10) Pub. No.: US 2021/0393950 A1
(43) Pub. Date: Dec. 23, 2021

(54) IMPLANTABLE APPARATUS FOR INTERACTING WITH BIOLOGICAL TISSUE

(71) Applicant: PANAXIUM, Aix en Provence (FR)

(72) Inventor: Christopher Michael PROCTOR, Port Washington, WI (US)

(73) Assignee: PANAXIUM, Aix en Provence (FR)

(21) Appl. No.: 17/290,104

(22) PCT Filed: Oct. 31, 2019
(86) PCT No.: PCT/IB2019/001282

§ 371 Date: Apr. 29, 2021

Related U.S. Application Data
(60) Provisional application No. 62/755,086, filed on Nov. 2, 2018.

(51) Int. Cl. A61N 1/05; A61N 2/02; A61N 5/06; A61N 2/00

(52) U.S. Cl. CPC A61N 1/0551; A61N 1/0529; A61N 2/02; A61N 5/0601; A61M 5/14276; A61N 5/0622; A61N 2005/0665; A61N 2005/0667; A61N 2/006

(57) ABSTRACT

There is described an implantable apparatus for interacting with biological tissue. The apparatus generally has a base; a plurality of strands each having an elongated body with a proximal end fixed to said base and a distal end opposite the said proximal end, the plurality of strands being entwined with one another to form a thread of entwined strands; and a plurality of biological interaction devices extending between the base and the plurality of strands, the biological interaction devices each having a biological interaction element located along the elongated body of a corresponding one of the plurality of strands and interacting with surrounding biological tissue when the thread of entwined strands is implanted into the biological tissue.

### FIELD

The improvements generally relate to apparatuses for implantation into biological tissue, and more specifically relate to apparatuses which interact with such biological tissue when so-implanted.

### BACKGROUND

An implantable apparatus may interact with the biological tissue into which it is implanted by sensing and/or stimulating the surrounding biological tissue. For instance, the implantable apparatus can interact with the biological tissue by sensing an electrical activity of the biological tissue. Additionally or alternatively, the implantable apparatus can interact with the biological tissue by electrically stimulating the biological tissue. Other forms of interaction may include electrochemical sensing or stimulation, optical sensing or stimulation, and sampling or delivery of biological materials including drugs, ions, molecules, metabolites, and other fluids. Although existing implantable apparatuses are satisfactory to a certain degree, there remains room for improvement.

### SUMMARY

It is often desirable for an implantable apparatus to include many biological interaction elements in order to increase the spatial resolution and/or control of the interaction. However, it can be difficult to fabricate such an implantable apparatus, especially when the implantable apparatus has a high density of such biological interaction elements. An implantable apparatus with many biological interaction elements may also be large and rigid, resulting in poor compatibility with the biological tissue into which it is implanted.

There is described an implantable apparatus for interacting with biological tissue. The implantable apparatus has a base, strands each having an elongated body with a proximal end fixed to the base and a distal end opposite the proximal end. As will be discussed below, the strands are entwined with one another to form a thread of entwined strands. For instance, the strands can be twisted with one another, braided with one another, woven with one another, self-assembled via capillary forces and/or surface tension forcing the strands to partially or wholly contact one another along their elongated bodies when pulled out of a liquid, or otherwise manipulated in any other suitable fashion. Biological interaction devices extending between the base and the entwined strands are also provided. Each biological interaction device has one or more biological interaction elements located along the elongated body of a corresponding strand which interacts with surrounding biological tissue when the thread of entwined strands is implanted into the biological tissue.

When the biological interaction devices of the implantable apparatus are interactingly coupled to an actuation apparatus, thereby forming a biological interaction system, biological tissue can be sensed and/or stimulated as desired. For instance, the actuation apparatus can sense electrical signal(s), optical signal(s), and/or biological fluid(s) originating from the biological tissue surrounding the implantable apparatus, and/or stimulate the surrounding biological tissue with electrical signal(s), optical signal(s), and/or external fluid(s) containing chemical(s), drug(s), molecule(s), metabolite(s), ion(s), water or any combination thereof.

In accordance with a first aspect of the present disclosure, there is provided an implantable apparatus for interacting with biological tissue, the apparatus comprising: a base; a plurality of strands each having an elongated body with a proximal end fixed to said base and a distal end opposite the said proximal end, the plurality of strands being entwined with one another to form a thread of entwined strands; and a plurality of biological interaction devices extending between the base and the plurality of strands, the biological interaction devices each having a biological interaction element located along the elongated body of a corresponding one of the plurality of strands and interacting with surrounding biological tissue when the thread of entwined strands is implanted into the biological tissue.

Further in accordance with the first aspect of the present disclosure, at least one of the plurality of biological interaction devices can for example have a conductive pad at the base, and a conductive trace running within the elongated body of a corresponding one of the plurality of strands and electrically connecting the biological interaction element of the at least one of the plurality of biological interaction devices to the conductive pad, the elongated body being made of an electrically insulating material.

Still further in accordance with the first aspect of the present disclosure, the biological interaction element can for example have at least one of an an electrode, a transistor, an electromagnetic coil, an antenna, an electrical transmitter, and an electrical detector.

Still further in accordance with the first aspect of the present disclosure, the electrically insulating material can for example be a polymeric material.

Still further in accordance with the first aspect of the present disclosure, at least one of the plurality of biological interaction devices can for example have an optical port at the base, an optical waveguide running within the elongated body of a corresponding one of the plurality of strands and optically connecting the biological interaction element of the at least one of the plurality of biological interaction devices to the optical port.

Still further in accordance with the first aspect of the present disclosure, the biological interaction element can for example have at least one of an opening along or at the end of the optical waveguide, a reflective surface, a focusing lens, a collecting lens and an optical filter.

Still further in accordance with the first aspect of the present disclosure, at least one of the plurality of biological interaction devices can for example have a microfluidic port at the base, a microfluidic channel running within the elongated body of a corresponding one of the plurality of strands and fluidically connecting the biological interaction element of the at least one of the plurality of biological interaction devices to the microfluidic port.

Still further in accordance with the first aspect of the present disclosure, the biological interaction element can for example have at least one of a fluidic entry port, a fluidic exit port, a nozzle and a valve.

Still further in accordance with the first aspect of the present disclosure, the elongated bodies of the plurality of strands can for example have a similar length.

Still further in accordance with the first aspect of the present disclosure, the entwined strands can for example be twisted with one another.

Still further in accordance with the first aspect of the present disclosure, the implantable apparatus can for example comprise one or more gaps within the entwined strands of the thread.

Still further in accordance with the first aspect of the present disclosure, the elongated bodies of the entwined strands of the thread can for example be in contact with one another.

Still further in accordance with the first aspect of the present disclosure, at least some of the plurality of biological interaction devices can for example extend between the base and one of the plurality of strands, with biological interaction elements being longitudinally spaced-apart from one another along the elongated body of the one of the plurality of strands.

Still further in accordance with the first aspect of the present disclosure, at least some of the biological interaction elements of the biological interaction devices of different strands can for example be longitudinally aligned with one another.

Still further in accordance with the first aspect of the present disclosure, said at least some of the biological interaction elements of the biological interaction devices can for example be located at the distal ends.

Still further in accordance with the first aspect of the present disclosure, said interacting can for example include at least one of electrically interacting, optically interacting and fluidically interacting with said surrounding biological tissue.

In accordance with a second aspect of the present disclosure, there is provided a method of interacting with biological tissue, the method comprising: entwining a plurality of strands to one another, thereby obtaining a thread of entwined strands each having an elongated body extending between a proximate end and an opposite distal end, a plurality of biological interaction elements being disposed along the elongated bodies of the entwined strands; implanting the thread of entwined strands within the biological tissue via the distal ends of the entwined strands of the thread; and the biological tissue surrounding the thread interacting with the plurality of biological interaction elements of the entwined strands.

Further in accordance with the second aspect of the present disclosure, said interacting for example comprise at least one of transmitting a signal to the surrounding biological tissue and receiving a signal from the surrounding biological tissue.

Still further in accordance with the second aspect of the present disclosure, said signal can for example be one of an electrical signal and an optical signal.

Still further in accordance with the second aspect of the present disclosure, said interacting can for example comprise at least one of injecting a fluid into the surrounding biological tissue and drawing a fluid from the surrounding biological tissue.

Still further in accordance with the second aspect of the present disclosure, said entwining can for example comprise at least one of twisting and braiding the plurality of strands with one another.

Still further in accordance with the second aspect of the present disclosure, said implanting can for example comprise implanting said thread of entwined strands into at least one of a central nervous system and a peripheral nervous system of a patient.

Still further in accordance with the second aspect of the present disclosure, the distal ends can for example be removably attached to a sacrificial member, said entwining comprising manipulating the sacrificial member relative to the proximal ends and removing the sacrificial member after said entwining.

The implantable apparatus is designed to address at least some shortcomings of some existing implantable apparatuses in terms of fabrication, implantation, ability to scale up the density of biological interaction elements, biocompatibility, and the ease of connectivity with any type of actuation apparatus, for instance. Broadly described, the implantable apparatus can be used for scientific, therapeutic, and/or diagnostic purposes including sensing electrical, optical, fluidic, metabolic, ionic, and/or chemical activity originating from cells, stimulation of cells in biological tissue, nervous system interfaces and brain-machine interfaces. More specifically, the implantable apparatus can be designed to sense or stimulate neural activity in cortex, hippocampus, or other areas of the brain to track activity of hundreds to thousands of neurons simultaneously or sequentially, individually or in groups, over a given period of time. The implantable apparatus can be designed to sense or stimulate neural activity in the peripheral nervous system as the thread of entwined strands can be sewn into a nerve fiber bundle to enable sensing and/or stimulating at a range of depths within the nerve fiber. The implantable apparatus can be designed to allow for sensing of specific metabolites or other biomarkers to allow for simultaneous or sequential sensing of one or more biomarkers in the surrounding biological tissue.

Many further features and combinations thereof concerning the present improvements will appear to those skilled in the art following a reading of the instant disclosure.

### DESCRIPTION OF THE FIGURES

In the figures,

FIG. 1 is a schematic view of an example of a biological interaction system adapted to electrically interact with surrounding biological tissue, in accordance with one or more embodiments;

FIG. 2A is a front elevation view of a plurality of strands being spaced-apart from one another, showing a biological interaction element along each strand, in accordance with one or more embodiments;

FIG. 2B is a front elevation view of the strands of FIG. 2A during entwining of the strands to one another;

FIG. 2C is a sectional view of the strands of FIG. 2B, taken along section 2C-2C of FIG. 2B;

FIG. 2D is a front elevation view of the entwined strands of FIG. 2B, forming a thread of entwined strands;

FIG. 2E is a sectional view of the thread of entwined strands of FIG. 2D, taken along section 2E-2E of FIG. 2D;

FIG. 3A is a front elevation view of a plurality of laterally spaced-apart strands, showing proximal ends of the strands being fixed to a base, and distal ends of the strands being fixed to a sacrificial member, in accordance with one or more embodiments;

FIG. 3B is a front elevation view of the strands of FIG. 3A being entwined with one another via a twisting of the sacrificial member;

FIG. 3C is a front elevation view of the entwined strands of FIG. 3B with the sacrificial member removed, forming a thread of entwined strands;

FIG. 4A is a front elevation view of a plurality of laterally spaced-apart strands, showing proximal ends of the strands being fixed to a base, and distal ends of the strands being immersed in a liquid, in accordance with one or more embodiments;

FIG. 4B is a front elevation view of the strands of FIG. 4A being entwined with one another as the strands are pulled out of the liquid, forming a thread of entwined strands;

FIG. 5A is a cross sectional view of a substrate on which lies a layer of electrically insulating material having a plurality of laterally spaced-apart conductive traces, in accordance with one or more embodiments;

FIG. 5B is a cross sectional view of the substrate of FIG. 5A, on which is deposited another layer of electrically insulating material;

FIG. 5C is a cross sectional view of the substrate of FIG. 5B, on which gaps are created between the spaced-apart conductive traces by removing portions of the electrically insulating material, resulting in spaced-apart strands;

FIG. 5D is a cross sectional view of the spaced-apart strands of FIG. 5C, showing biological interaction elements on some of the strands and the substrate being removed thereby freeing the strands from the substrate and from one another;

FIG. 6A is cross sectional view of an example of a strand, showing a conductive trace extending within the strand, in accordance with one or more embodiments;

FIG. 6B is cross sectional view of another example of a strand, showing a plurality of horizontally spaced-apart conductive traces within the strand, in accordance with one or more embodiments;

FIG. 6C is cross sectional view of another example of a strand, showing a plurality of vertically spaced-apart conductive traces within the strand, in accordance with one or more embodiments;

FIG. 7 is an image of an example of an implantable apparatus, showing strands fixed to a base, in accordance with one or more embodiments;

FIG. 7A is an enlarged view of the strands of the implantable apparatus of FIG. 7, showing the strands being entwined with one another;

FIG. 7B is an enlarged view of the entwined strands of the implantable apparatus of FIG. 7, taken at inset 7B of FIG. 7A;

FIG. 8 is an image of an example of a thread of entwined strands coated with a bioresorbable shuttle for implantation into biological tissue, in accordance with one or more embodiments;

FIG. 9A is an image of an example of a thread of entwined strands being attached to a needle shuttle for implantation into biological tissue, in accordance with one or more embodiments;

FIG. 9B is an image of the thread of entwined strands of FIG. 9A being implanted into the biological tissue after the removal of the needle shuttle;

FIG. 10 is a schematic view of an example of a biological interaction system adapted to optically interact with surrounding biological tissue, in accordance with one or more embodiments; and

FIG. 11 is a schematic view of an example of a biological interaction system adapted to fluidically interact with surrounding biological tissue, in accordance with one or more embodiments.

### DETAILED DESCRIPTION

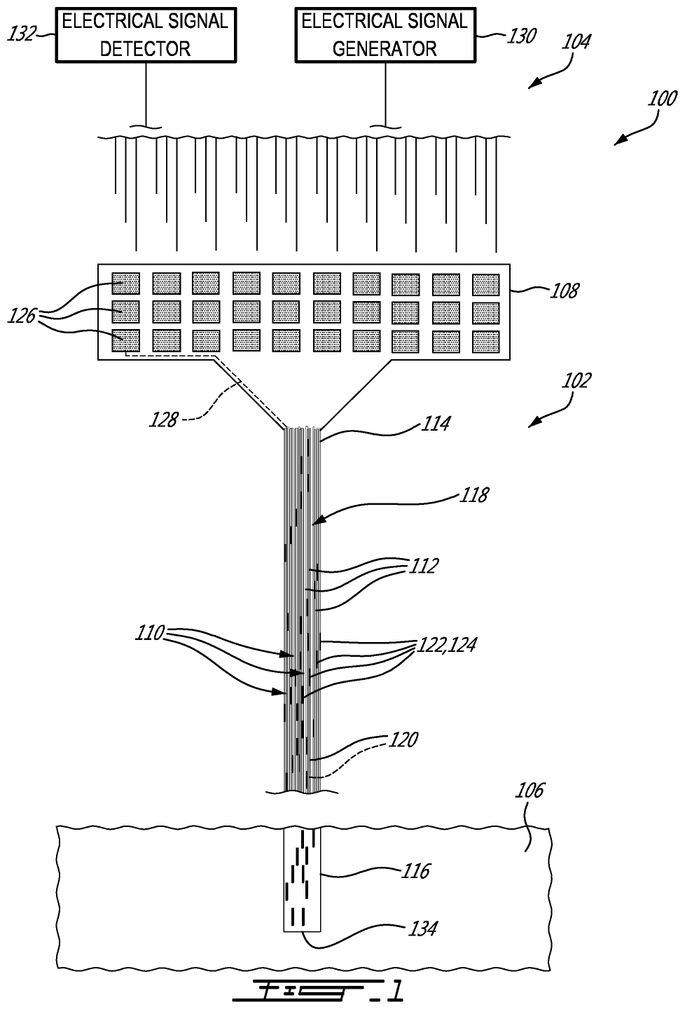

FIG. 1 is an example of a biological interaction system 100, in accordance with an embodiment. As depicted, the biological interaction system 100 has an implantable apparatus 102 and an actuation apparatus 104 which is interactingly coupled to the implantable apparatus 102. As will be described below the biological interaction system 100 is adapted to electrically interact with biological tissue 106 surrounding at least a portion of the implantable apparatus 102 when it is implanted into the biological tissue 106. For instance, the biological interaction system 100 can sense electrical activity of the surrounding biological tissue 106 and/or electrically stimulate the surrounding biological tissue 106.

As illustrated, the implantable apparatus 102 has a base 108, and strands 110 fixed to the base 108 at an end thereof. More specifically, each strand 110 has an elongated body 112 with a proximal end 114 fixed to the base 108 and a distal end 116 opposite to the proximal end 114. As shown, the strands 110 are entwined with one another to form a thread 118 of entwined strands 110. The thread 118 of entwined strands 110 thereby has a fixed end and an opposite free end to be implanted in the biological tissue 106. The thread 118 of entwined strands 110 can be designed to be biocompatible, and also flexible with cellular-size dimensions to reduce foreign-body response.

The base can take many forms. In some embodiments, the base can be flat, with the strands all connected to one or more edges of the flat base. In some other embodiments, the base can have a cube-like body, with the strands connected to one or more sides of the cube-like body in a two dimensional array. Other embodiments may apply.

It is intended that the thread 118 of entwined strands 110 can have one or more gaps 120 separating some portions of two or more adjacent strands 110, which may advantageously increase the contact surface with the biological tissue 106 and bodily fluids, as fluids and cells can move into and through the thread 118 via the gaps 120. Moreover, it is noted that the thread 118 of entwined strands 110 can be more flexible than existing implantable apparatuses with the same number of biological interaction elements thanks to at least part of the gaps 120 between the entwined strands 110. In some other embodiments, the elongated bodies 112 of the strands 110 may be in contact with one another along their entire lengths. Accordingly, the density of the thread 118 of entwined strands 110 can be adjusted to allow both densely packed threads as well as loosely packed threads, i.e., structures with physical space between the strands 110 that may be equal to one or more strands. The implantable apparatus 102 can also be configured to allow for expansion of the thread 118 once it is implanted into the biological tissue 106 in order to allow for the penetration of cellular structures and other biological media. In any case, the thread 118 of entwined strands 110 can be formed such that it maintains its original form following implantation.

The number of strands 110 can vary from one embodiment to another. However, in some embodiments, the implantable apparatus 102 can have at least two strands 110, at least ten strands 110, or more, and so forth, depending on the embodiment. The strands 110 may be straight or with curves and turns in some embodiments. The length of the entwined strands 110 can vary as well. For instance, the entwined strands 110 of the thread 118 can share a similar length to one another in some embodiments whereas the entwined strands 110 of the thread 118 can have different lengths in some other embodiments. In some embodiments, the length of the strands 110 can range between 1 mm and 1 m, preferably between 5 cm and 500 cm, and most preferably between 1 cm and 10 cm. The strands 110 can be thin, with widths and depths each varying between 0.1 μm and 1 mm, preferably between 25 μm and 500 μm and most preferably between 50 μm and 100 μm. The strands 110 can have cross-sectional areas varying between 1 μm² and 1 cm², preferably between 10 μm² and 0.9 cm², and most preferably between 100 μm² and 0.5 cm². It was found that in embodiments where the width of the thread 118 of entwined strands 110 corresponds to the cell dimension associated to the biological tissue, rejection of the implantable apparatus 102 may be less likely.

As shown in the depicted embodiment, biological interaction devices 122 extend between the base 108 and the strands 110. Each biological interaction device 122 has one or more biological interaction elements 124 located along the elongated body 112 of a corresponding strand 110 of the thread 118. The biological interaction elements 124 are configured to interact with surrounding biological tissue 106 when the thread 118 of entwined strands 110 is implanted into the biological tissue 106.

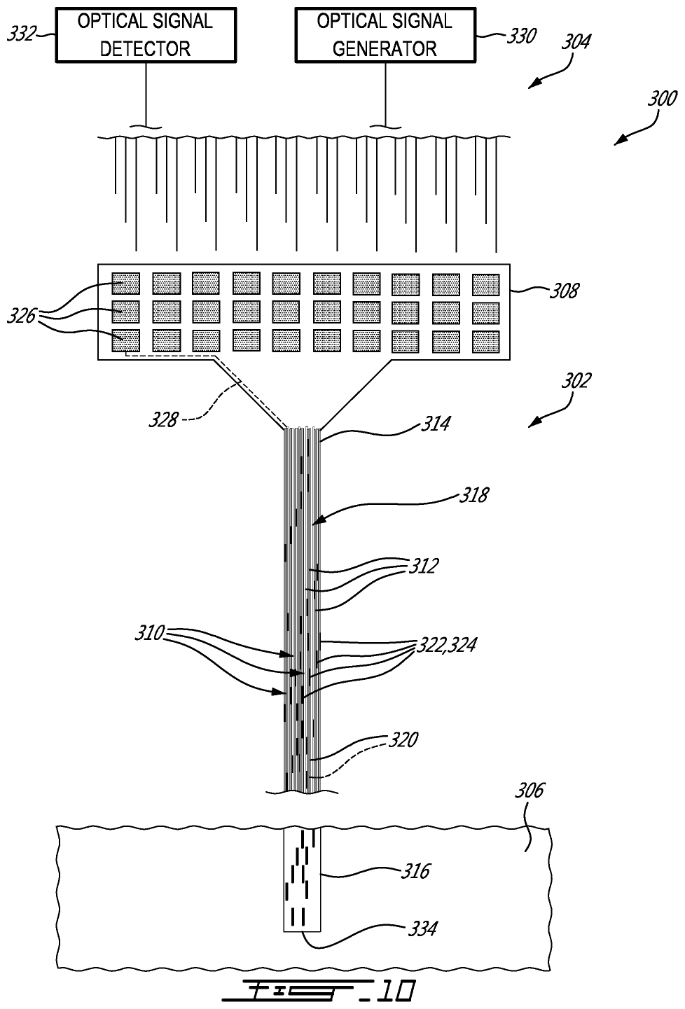

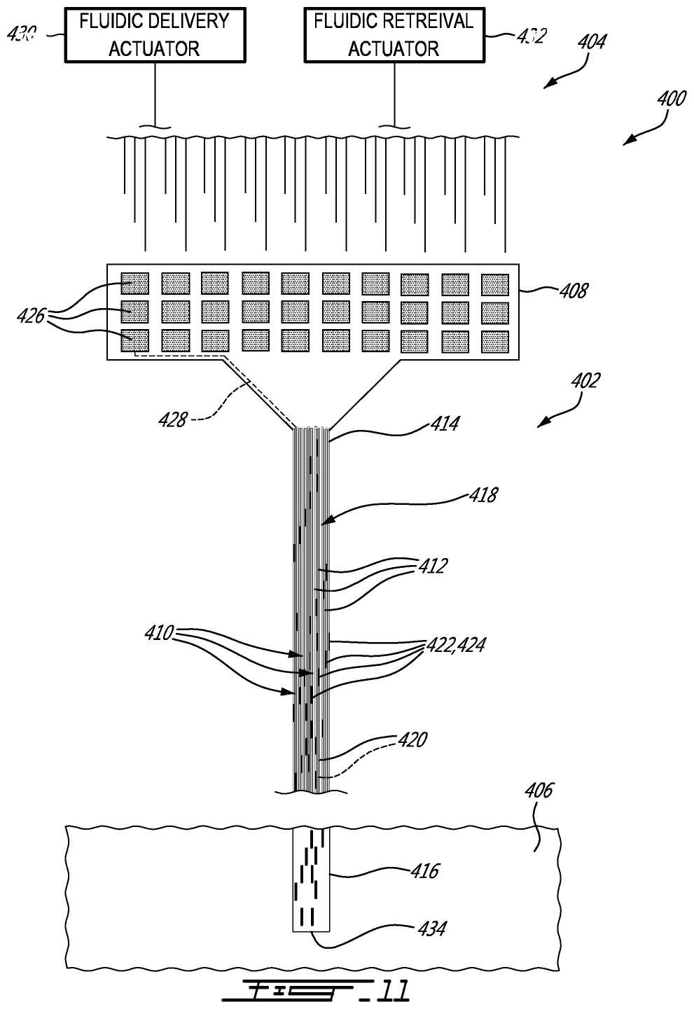

In this specific embodiment, the interaction between the implantable apparatus 102 and the biological tissue 106 is of electrical nature, as the biological interaction devices 122 are configured for sensing electrical activity of the surrounding biological tissue 106 and/or electrically stimulating the surrounding biological tissue 106. However, as described below with reference to FIGS. 10 and 11, the interaction between some other example implantable apparatuses and the biological tissue 106 can involve, additionally or alternately, an optical, fluidic and/or chemical component(s). It is thereby envisaged that the interaction between the implantable apparatus 102 and the biological tissue 106 can include one or more of the following types of interaction: electrical, optical, fluidic and chemical. In any case, the interaction between the biological interaction elements 124 and the biological tissue 106 can be performed in a simultaneous manner among the biological interaction elements 124 or sequentially, one biological interaction element 124 after the other.

Still referring to FIG. 1, in this specific embodiment, the biological interaction devices 122 each have a conductive pad 126 at the base 108, and one or more conductive traces 128 running within the elongated body 112 of a corresponding strand 110, thereby electrically connecting the biological interaction element 124 of the corresponding strand 110 to the corresponding conductive pad 126 at the base 108. In such embodiments, the elongated bodies 112 of the strands 110 are made of an electrically insulating and biocompatible material, thereby preventing any undesired electrical interaction between the conductive traces 128 and the biological tissue 106 surrounding the strands 110. An example of such an electrically insulating and biocompatible material can include, but not limited to, polymeric material such as parylene, SU8, PDMS or polyurethane. Different biological interaction devices 124 can share one or more conductive pads 126 at the base 108.

It is envisaged that the electrical interaction with the biological tissue 106 may be performed only via the biological interaction elements 124. For instance, the biological interaction elements 124 can include an electrode, a transistor, an electromagnetic coil or antenna, or some other electrical transmitter or detector. In some embodiments, the transistor can be a regular transistor for sensing or delivering electrical activity. However, in some other embodiments, the transistor can be configured to sense chemical activity, for example by coating an exposed surface of the transistor with one or more chemically sensitive materials or enzymes. Additionally or alternately, the biological interaction elements 124 can have a circular shape, a rectangular shape, or any other suitable shape, with dimensions suitable for measuring single cell activity (e.g., 10 μm×10 μm) or for interacting with groups of a few or many cells together.

The type of electrical components included in each biological interaction element 124 can differ from one biological interaction device 122 to another. For instance, some of the biological interaction elements 124 can include electrical transmitters whereas some other of the biological interaction elements 124 can include electrical detectors. It is intended that the type of electrical components of the biological interaction elements 124 dictate the construction of the actuation apparatus 104.

In the illustrated embodiment, the actuation apparatus 104 has an electrical signal generator 130 which is electrically connected to at least some of the electrical pads 126 at the base 108, and an electrical signal detector 132 which is electrically connected to some other of the electrical pads 126 at the base 108. Some or all of the electrical pads 126 at the base 108 may also be connected to both the electrical signal generator 130 and the electrical signal detector 132 for bidirectional interaction with the biological tissue. In such an embodiment, the biological tissue 106 may be electrically stimulated by the electrical signal generator 130 generating an electrical signal to be propagated along some of the conductive traces 128 via the electrical pads 126, before or after which, or at the same time as, the biological interaction elements 124 may transmit corresponding electrical signals to the surrounding biological tissue 106. Understandably, the electrical activity of the surrounding biological tissue 106 may be picked up by the biological interaction elements 124 only to be communicated to the electrical signal detector 132 via the conductive traces 128 and the electrical pads 126 for sensing purposes. Wires of the actuation apparatus 104 can be electrically connected to the electrical pads 126 using simultaneous electrical connection methods, including, but not limited to, zero-insertion-force connectors, flexible ribbon cable bonding, and the like.

Of course, the actuation apparatus 104 can differ from the one illustrated in FIG. 1. For instance, the actuation apparatus 104 may comprise a computing device having a processor and a computable-readable non-transitory memory having instructions that when executed by the processor perform one or more steps to interact with the biological tissue 106 via the implantable apparatus 102. In some embodiments, the actuation apparatus 104 can have a wired and/or wireless connection with the implantable apparatus 102. The actuation apparatus 104 may be made integral to the base 108 of the implantable device 102.

The biological interaction element(s) 124 can be positioned at any longitudinal position along the elongated body 112 of a strand 110. Additionally or alternatively, the biological interaction elements 124 of different strands 110 may be longitudinally aligned with or spaced-apart from one another. For instance, in some embodiments, the biological interaction element 124 may be located at the distal ends 116 of the elongated bodies 112 of the strands 110, thereby providing electrical interaction at a tip 134 of the thread 118.

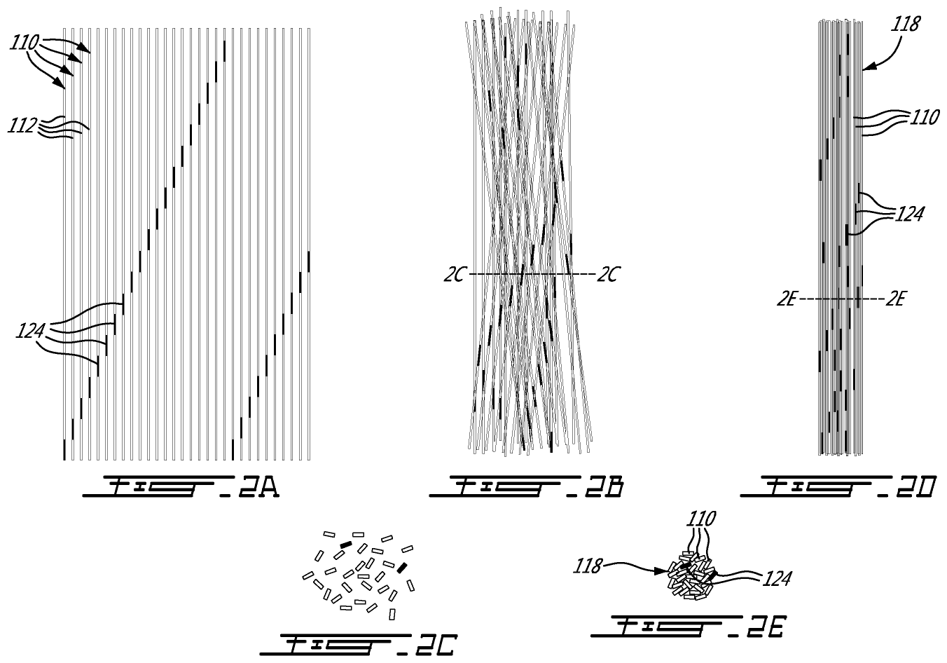

FIGS. 2A-2E show exemplary laterally spaced-apart strands 110 having biological interaction elements 124 being longitudinally spaced-apart from one another. More specifically, as shown in FIG. 2A, strands 110 can be first positioned in a laterally spaced-apart manner with each strand 110 being parallel to the other strands 110. In this specific embodiment, each strand 110 has a single biological interaction element 124 located at some longitudinal position along the elongated body 112 of the strand 110. In this way, when the strands 110 are entwined with one another, such as shown in FIGS. 2B and 2C, to form the thread 118 of entwined strands 110 of FIGS. 2D and 2E, the biological interaction elements 124 can appear as being evenly or randomly distributed from one another along the length of the thread 118. As best shown in FIGS. 2C and 2E, the positioning of the biological interaction elements 124 along the strands 110 shown in this example cause the thread 118 of entwined strands 110 to have on average at least two biological interaction elements 124 at any point along the long-axis. In this specific embodiment, the probability that at least one of the biological interaction elements 124 will be at least partially exposed to the surrounding biological tissue 106 can increase.

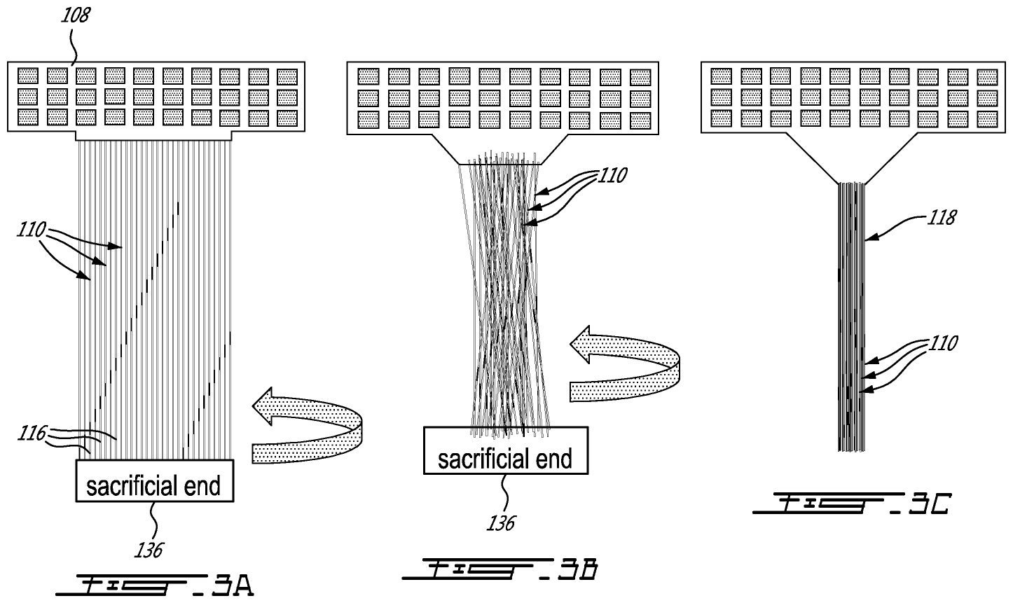

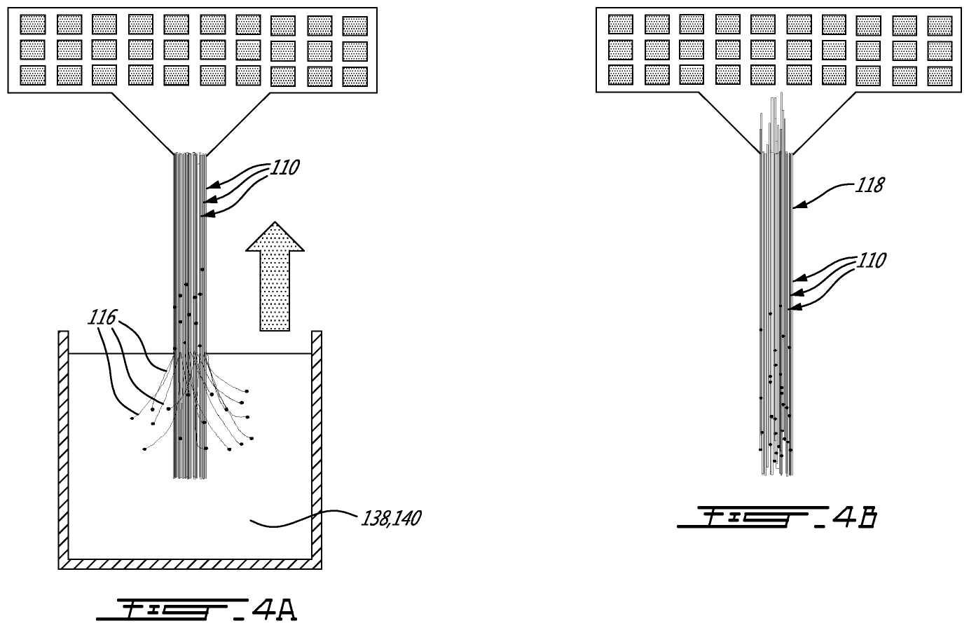

The stands 110 may be entwined with one another in any suitable manner. For instance, in some embodiments, the strands 110 are entwined with one another by twisting the strands 110 to one another. In these embodiments, one or more sacrificial members 136 may be attached to the distal ends 116 of the strands 110 such as shown in FIG. 3A. The entwining can be performed using the sacrificial member(s). For instance, cy rotating the sacrificial member 136, and/or the base 108, about the lengths of the strands 110, such as shown in FIG. 3B, the strands 110 can be entwined in a straightforward manner. Once the strands 110 as so-entwined with one another, the sacrificial member 136 can be removed, thereby freeing the distal ends 116 of the entwined strands 110 to form the thread 118 shown in FIG. 3C. In some other embodiments, the sacrificial member 136 is optional. For instance, in some embodiments, the strands 110 are self-entwined with one another by immersing the strands 110 into a liquid 138, such as water 140, which when pulled away from the liquid 138, will force the strands 110 to partially or wholly entwine with one another thanks to capillary forces and surface tension exerted within the liquid 116 as shown in FIG. 4A, to ultimately result in the thread 118 of entwined strands 110 shown in FIG. 4B. Alternately or additionally, the strands 110 may be braided or woven with one another in some embodiments. It is noted that the distal ends 116 of the strands 110 may each be attached individually or in groups to a different sacrificial member to facilitate this braiding or weaving before the sacrificial members are removed.

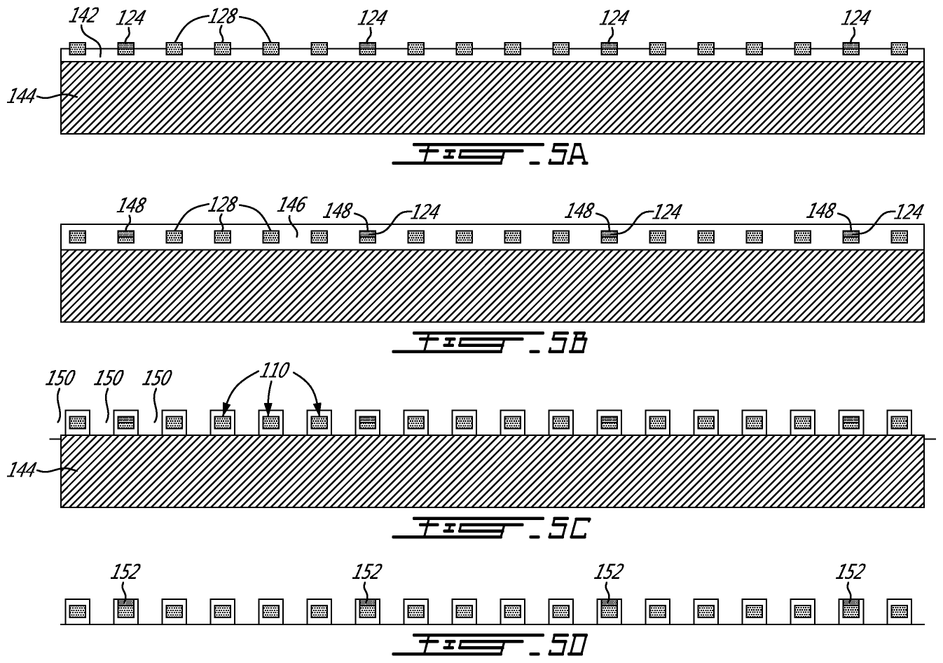

The implantable apparatus can be fabricated using a variety of approaches, including standard microfabrication techniques. FIGS. 5A-5D illustrate an exemplary method of fabricating the strands 110. As shown in FIG. 5A, a first layer 142 of electrically insulating material is deposited on a substrate 144. As depicted, a plurality of laterally spaced-apart conductive traces 128 and biological interaction elements 124 are patterned on the first layer 142 of electrically insulating material. Then, a second layer 146 of electrically insulating material is deposited on the conductive traces 128, such as shown in FIG. 5B, to insulate the conductive traces 128 everywhere except for openings 148 at the electrical pads and the biological interaction elements 124. As shown in FIG. 5C, gaps 150 between the laterally spaced-apart conductive traces 128 are created by removing portions of the first and second layers 142 and 146 of electrically insulating material, thereby leaving a corresponding plurality of strands 110 lying on the substrate 144. As shown in this latter figure, a conducting polymer 152 or other impedance reducing material can be deposited on the openings 148 to cover the biological interaction elements 124. The deposition of the conducting polymer 152 can alternatively be done earlier in the process, for example before depositing the second layer 146 of insulating material, or before creating the gaps 150 by removing some of the insulating material. By removing the substrate 144 as shown in FIG. 5D, the plurality of strands 110 can be freed for later entwinement.

The openings 148 at the electrical pads and biological interaction elements 124 can be made by selective deposition of the insulating material (e.g., photolithography, printing, chemical vapor deposition), by selective removal of the insulating material (e.g., wet or dry etching, laser ablation), or by some combination of the two. The electrically insulating material between the conductive traces 128 for the portion of the implantable apparatus that forms the strands 110 would be removed by a similar means either concurrently or subsequently. Such a fabrication can be done such that a thin layer of insulation is maintained on all sides of the conductive traces 128 within a strand 110. A thin coating of bioresorbable or other material may be applied to the thread 118 of entwined strands 110 at any point in the above methods in order to help maintain the desired formation. Other methods of fabricating the strands 110 can be performed in some other circumstances.

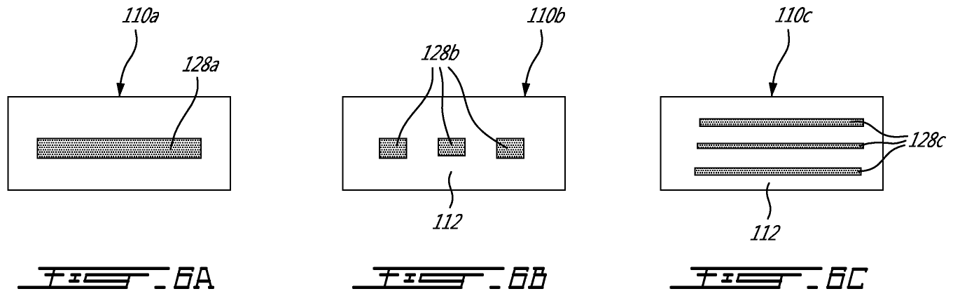

In some embodiments, each strand 110 may have one or more biological interaction elements 124. FIG. 6A shows a cross section of a strand 110a having only one conductive trace 128a leading to a single biological interaction element. In the case where more than one biological interaction elements may be incorporated to a single strand, the biological interaction elements may be longitudinally spaced-apart from one another along the elongated body of the single strand. The biological interaction elements may be evenly or unevenly distributed along the strand, depending on the embodiment, and the conductive traces may be laterally spaced-apart from one another within the elongated body of the strand. FIGS. 6B and 6C show cross sections of exemplary strands 110b and 110c having a plurality of conductive trace 128 extending within. More specifically, FIG. 6B shows three conductive traces 128b being vertically stacked and spaced-apart from one another within the elongated body 112 whereas FIG. 6C shows three conductive traces 128c being horizontally stacked and spaced-apart from one another within the elongated body 112. Conductive traces 128 may also be stacked and spaced apart both vertically and horizontally.

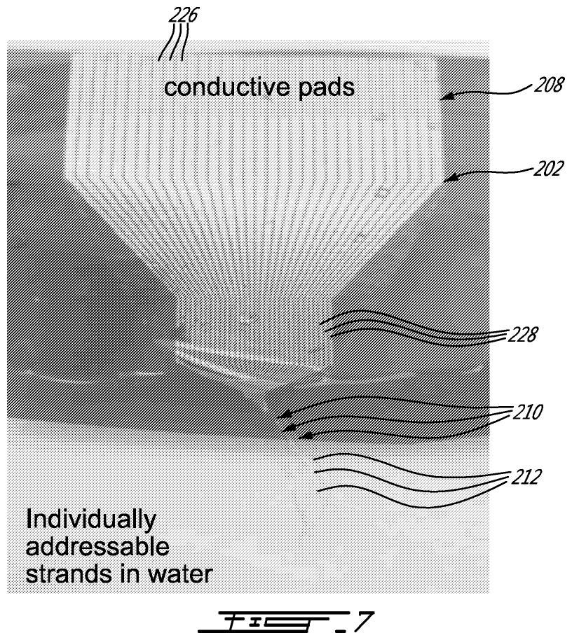

FIG. 7 shows another example of an implantable apparatus 202, in accordance with another embodiment. As depicted, the implantable apparatus 202 has a base 208 on which spaced-apart conductive pads 226 lie. In this embodiment, the conductive pads 226 are fixed on the base 208. As shown, the conductive traces 228 taper and get closer to one another as the conductive traces 228 extend away from the base 208. The conductive traces 228 extend within the elongated bodies 212 of the strands 210 which are made of an electrically insulating material. FIGS. 7A and 7B show enlarged views of a thread 218 made of the entwined strands 210 of FIG. 7. As shown, the thread 218 is formed of a plurality of strands 210 entwined with one another, and has a width of about 90 pm in this embodiment.

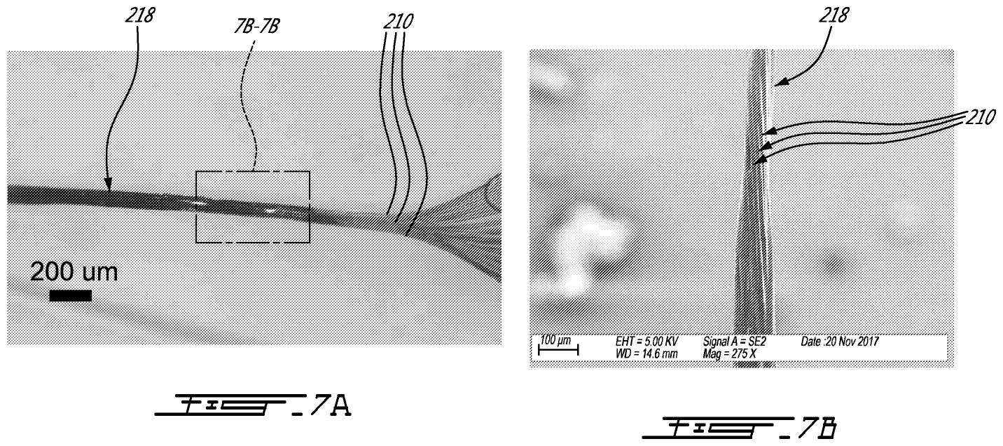

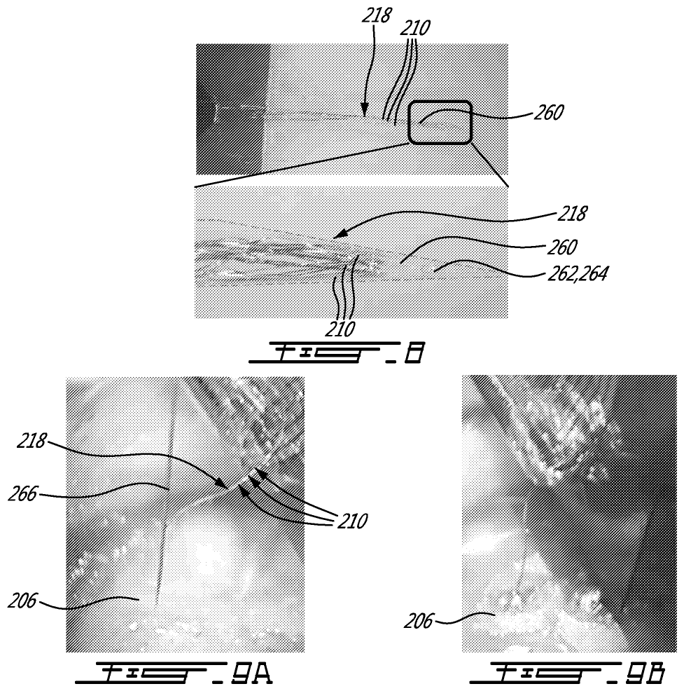

The method of implanting the implantable apparatus 202 into biological tissue can differ from one embodiment to another. The implantable apparatus 202 can be implanted into the biological tissue directly without the need for a shuttle or other indirect implanting means. In some embodiments, FIG. 8 shows an example where the thread 218 of entwined strands 210 is received in a shuttle 260 of bioresorbable structural material. In this example, the shuttle 260 can be forced into biological tissue. However, after some time, the bioresorbable nature of the shuttle 260 causes the shuttle 260 to dissipate, thereby leaving the thread 218 of entwined strands 210 within the biological tissue. The thread 218 of entwined strands 210 can additionally or alternatively be covered in some bioresorbable material 262 which is sized and shaped to form a tip 264 for penetrating in the biological tissue in some embodiments. In another example, as shown in FIGS. 9A and 9B, the thread 218 of entwined strands 210 is provisionally attached to a needle shuttle 266 (e.g., stiff shuttle, pin, microneedle, hollow tube) for implantation into the biological tissue 206 in a manner analogous to sewing with a thread and needle. FIG. 9A shows the thread 218 being implanted using the needle shuttle 266 whereas FIG. 9B shows the implanted thread 218 after the needle shuttle 266 has been removed. It is envisaged that the shuttle 266 can stay in the biological tissue 206 or be removed via the original entry path or an alternative one, leaving behind only the thread 218 of entwined strands 210 into the biological tissue 206.

FIG. 10 is an example of a biological interaction system 300, in accordance with an embodiment. As depicted, the biological interaction system 300 has an implantable apparatus 302 and an actuation apparatus 304 which is interactingly coupled to the implantable apparatus 302. In this specific embodiment, the biological interaction system 300 is adapted to optically interact with biological tissue 306 surrounding at least a portion of the implantable apparatus 302 when it is implanted into the biological tissue 306. For instance, the biological interaction system 300 can sense optical activity of the surrounding biological tissue 306 and/or optically stimulate the surrounding biological tissue 306.

As illustrated, the implantable apparatus 302 has a base 308, and strands 310 fixed to the base 308 at an end thereof. More specifically, each strand 310 has an elongated body 312 with a proximal end 314 fixed to the base 308 and a distal end 316 opposite to the proximal end 314. As shown, the strands 310 are entwined with one another to form a thread 318 of entwined strands 310. The thread 318 of entwined strands 310 thereby has a fixed end and an opposite free end to be implanted in the biological tissue 306. In this specific embodiment, the biological interaction devices 322 each have an optical port 326 at the base 308, and an optical waveguide 328 running within the elongated body 312 of a corresponding strand 310, thereby optically connecting the biological interaction element 324 of the corresponding strand 310 to the corresponding optical port 326 at the base 308. Examples of such optical waveguide 328 can include, but not limited to, fiber(s), strip waveguide(s) and the like. In such embodiments, the elongated bodies 312 of the strands 310 are made of a material preventing any undesired optical interaction between the optical waveguides 328 and the biological tissue 306 surrounding the strands 310.

It is envisaged that the optical interaction with the biological tissue 306 may be performed only via the biological interaction elements 324. For instance, the biological interaction elements can include an opening along or at the end of optical waveguide 328, a reflective surface, a focusing lens, a collecting lens and an optical filter.

The type of electro-optical components included in each biological interaction element 324 can differ from one biological interaction device 322 to another. For instance, some of the biological interaction elements 324 can include optical transmitters whereas some other of the biological interaction elements 324 can include electrical detectors. It is intended that the type of electro-optical components of the biological interaction elements 324 dictate the construction of the actuation apparatus 304.

In the illustrated embodiment, the actuation apparatus 304 has an optical signal generator 330 which is optically connected to at least some of the optical ports 326 at the base 308, and an optical signal detector 332 which is optically connected to some other of the optical ports 326 at the base 308. Some or all of the electrical pads 326 at the base 308 may also be connected to both the optical signal generator 330 and the optical signal detector 332. In such an embodiment, the biological tissue 306 may be optically stimulated by the optical signal generator 330 generating an optical signal to be propagated along some of the optical waveguides 328 via the optical ports 326, before or after which, or as the same time as, the biological interaction elements 324 may transmit corresponding optical signals to the surrounding biological tissue 306. Understandably, the optical activity of the surrounding biological tissue 306 may be picked up by the biological interaction elements 324 only to be communicated to the optical signal detector 332 via the optical waveguides 328 and the optical ports 326 for sensing.

FIG. 11 is an example of a biological interaction system 400, in accordance with an embodiment. As depicted, the biological interaction system 400 has an implantable apparatus 402 and an actuation apparatus 404 which is interactingly coupled to the implantable apparatus 402. In this specific embodiment, the biological interaction system 402 is adapted to fluidically interact with biological tissue 406 surrounding at least a portion of the implantable apparatus 402 when it is implanted into the biological tissue 406. For instance, the biological interaction system 400 can inject fluid into the surrounding biological tissue 406 and/or draw fluid from the surrounding biological tissue 406.

As illustrated, the implantable apparatus 402 has a base 408, and strands 410 fixed to the base 408 at an end thereof. More specifically, each strand 410 has an elongated body 412 with a proximal end 414 fixed to the base 408 and a distal end 416 opposite to the proximal end 414. As shown, the strands 410 are entwined with one another to form a thread 418 of entwined strands 410. The thread 418 of entwined strands 410 thereby has a fixed end and an opposite free end to be implanted in the biological tissue 406. In this specific embodiment, the biological interaction devices 422 each have a fluidic port 426 at the base 408, and a microfluidic channel 428 running within the elongated body 412 of a corresponding strand 410, thereby fluidically connecting the biological interaction element 424 of the corresponding strand 410 to the corresponding fluidic port 426 at the base 408. In some embodiments, there can be several biological interaction elements 424 sharing a single microfluidic channel 428 and/or sharing a fluidic port 426 at the base 408. Examples of such microfluidic channels 428 can have, but not limited to, a circular cross-section, a rectangular cross-section and the like. In such embodiments, the elongated bodies 412 of the strands 410 are made of a material preventing any undesired fluidic interaction between the microfluidic channels 428 and the biological tissue 406 surrounding the strands 410.

In addition, in some embodiments, the biological interaction devices 422 are configured to control the biological interaction elements 424. For instance, to open and/or close one or more fluidic ports of the strands 410. Accordingly, the biological interaction devices 422 can have a combination of fluidic channels 428 for the fluid exchange and of electrical traces for communicating control signals.

It is envisaged that the fluidic interaction with the biological tissue 406 may be performed only via the biological interaction elements 424. For instance, the biological interaction elements can include a fluidic entry port, a fluidic exit port, a nozzle and a valve.

The type of fluidic components included in each biological interaction element 424 can differ from one biological interaction device 422 to another. For instance, some of the biological interaction elements 424 can include fluid pushing nozzles whereas some other of the biological interaction elements 424 can include fluid drawing nozzles. It is intended that the type of fluidic components of the biological interaction elements 424 dictate the construction of the actuation apparatus 404.

In the illustrated embodiment, the actuation apparatus 404 has a number of fluidic pumps, either acting as fluidic delivery or fluidic retrieval actuators 430 and 432, e.g., syringe pumps, which are fluidically connected to at least some of the fluidic ports 426 at the base 408. The fluidic pumps can push fluid into the biological tissue 406 and/or draw fluid from the biological tissue 406, depending on the embodiment. Some or all of the fluidic port 426 at the base 408 may also be connected to both the fluidic delivery and fluidic retrieval actuators 430 and 432. In such an embodiment, the biological tissue 406 may be fluidicly stimulated by the fluidic delivery actuator 430 pushing an external fluid to be conveyed along some of the microfluidic channels 428 via the fluidic ports 426, before or after which, or at the same time as, the biological interaction elements 424 may convey corresponding external fluid to the surrounding biological tissue 406. In some embodiments, the external fluid can contain chemical(s), drug(s), molecule(s), metabolite(s), ion(s), water or any combination thereof. Understandably, the fluidic activity of the surrounding biological tissue 406 may be picked up by the biological interaction elements 424 only to be retrieved by the fluidic retrieval actuators 432 via the microfluidic channels 428 and the fluidic ports 426 for sensing purposes.

As can be understood, the examples described above and illustrated are intended to be exemplary only. The scope is indicated by the appended claims.

1. An implantable apparatus for interacting with biological tissue, the apparatus comprising:
a base;
a plurality of strands each having an elongated body with a proximal end fixed to said base and a distal end opposite the said proximal end, the plurality of strands being entwined with one another to form a thread of entwined strands; and
a plurality of biological interaction devices extending between the base and the plurality of strands, the biological interaction devices each having a biological interaction element located along the elongated body of a corresponding one of the plurality of strands and interacting with surrounding biological tissue when the thread of entwined strands is implanted into the biological tissue.

2. The implantable apparatus of claim 1 wherein at least one of the plurality of biological interaction devices has a conductive pad at the base, and a conductive trace running within the elongated body of a corresponding one of the plurality of strands and electrically connecting the biological interaction element of the at least one of the plurality of biological interaction devices to the conductive pad, the elongated body being made of an electrically insulating material.

3. The implantable apparatus of claim 2 wherein the biological interaction element has at least one of an an electrode, a transistor, an electromagnetic coil, an antenna, an electrical transmitter, and an electrical detector.

4. The implantable apparatus of claim 2 wherein the electrically insulating material is a polymeric material.

5. The implantable apparatus of claim 1 wherein at least one of the plurality of biological interaction devices has an optical port at the base, an optical waveguide running within the elongated body of a corresponding one of the plurality of strands and optically connecting the biological interaction element of the at least one of the plurality of biological interaction devices to the optical port.

6. The implantable apparatus of claim 5 wherein the biological interaction element has at least one of an opening along or at the end of the optical waveguide, a reflective surface, a focusing lens, a collecting lens and an optical filter.

7. The implantable apparatus of claim 1 wherein at least one of the plurality of biological interaction devices has a microfluidic port at the base, a microfluidic channel running within the elongated body of a corresponding one of the plurality of strands and fluidically connecting the biological interaction element of the at least one of the plurality of biological interaction devices to the microfluidic port.

8. (canceled)

9. The implantable apparatus of claim 1 wherein the elongated bodies of the plurality of strands have a similar length.

10. The implantable apparatus of claim 1 wherein the entwined strands are twisted with one another.

11. The implantable apparatus of claim 1 further comprising one or more gaps within the entwined strands of the thread.

12. The implantable apparatus of claim 1 wherein the elongated bodies of the entwined strands of the thread are in contact with one another.

13. The implantable apparatus of claim 1 wherein at least some of the plurality of biological interaction devices extend between the base and one of the plurality of strands, with biological interaction elements being longitudinally spaced-apart from one another along the elongated body of the one of the plurality of strands.

14. The implantable apparatus of claim 1 wherein at least some of the biological interaction elements of the biological interaction devices of different strands are longitudinally aligned with one another.

15. (canceled)

16. The implantable apparatus of claim 1 wherein said interacting is at least one of electrically interacting, optically interacting and fluidically interacting with said surrounding biological tissue.

17. A method of interacting with biological tissue, the method comprising:
entwining a plurality of strands to one another, thereby obtaining a thread of entwined strands each having an elongated body extending between a proximate end and an opposite distal end, a plurality of biological interaction elements being disposed along the elongated bodies of the entwined strands;
implanting the thread of entwined strands within the biological tissue via the distal ends of the entwined strands of the thread; and
the biological tissue surrounding the thread interacting with the plurality of biological interaction elements of the entwined strands.

18. The method of claim 17 wherein said interacting comprises at least one of transmitting a signal to the surrounding biological tissue and receiving a signal from the surrounding biological tissue.

19. (canceled)

20. The method of claim 17 wherein said interacting comprises at least one of injecting a fluid into the surrounding biological tissue and drawing a fluid from the surrounding biological tissue.

21. The method of claim 17 wherein said entwining comprises at least one of twisting and braiding the plurality of strands with one another.

22. The method of claim 17 wherein said implanting comprises implanting said thread of entwined strands into at least one of a central nervous system and a peripheral nervous system of a patient.

23. The method of claim 17 wherein the distal ends being removably attached to a sacrificial member, said entwining comprising manipulating the sacrificial member relative to the proximal ends and removing the sacrificial member after said entwining.
